(19) United States
(12) Patent Application Publication
(10) Pub. No.: US 2020/0194682 A1
(43) Pub. Date: Jun. 18, 2020

(54) THERMALLY ACTIVATED DELAYED FLUORESCENCE MATERIAL AND METHOD FOR PREPARING THEREOF AND ORGANIC ELECTROLUMINESCENT DIODE DEVICE

(71) Applicant: Wuhan China Star Optoelectronics Semiconductor Display Technology Co., Ltd., Wuhan (CN)

(72) Inventor: Jiajia LUO, Wuhan (CN)

(73) Assignee: Wuhan China Star Optoelectronics Semiconductor Display Technology Co., Ltd., Wuhan (CN)

(21) Appl. No.: 16/470,552

(22) PCT Filed: Feb. 20, 2019
(86) PCT No.: PCT/CN2019/075616

§ 371 Date: Jun. 18, 2019

(30) Foreign Application Priority Data
Dec. 17, 2018 (CN) 201811545490.8

(51) Int. Cl. H01L 51/00; C09K 11/06; C07D 219/14; C07D 265/38; C07D 279/26

(52) U.S. Cl. CPC H01L 51/0071; C09K 11/06; C07D 219/14; H01L 51/5016; C07D 265/38; C07D 279/26; C09K 2211/1018; H01L 51/0072

(57) ABSTRACT

A thermally activated delayed fluorescent (TADF) compound, a method for preparing thereof, and an organic electroluminescent diode device are provided. The thermally activated delayed fluorescent compound includes a chemical structure of formula I:

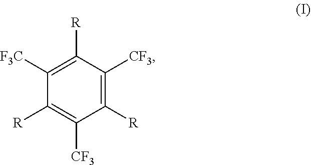

and R is an electron donating group. A trifluoromethyl group is used as a strong electron acceptor group, and an electron donor group is modified by combining different functional groups. An influence of the strength of the electron donor on material properties is researched to design a blue-light thermal activation delayed fluorescent compounds with significant TADF properties. The thermally activated delayed fluorescent compounds have a high reaction rate constant of reverse intersystem enthalpy constant (k_{RISC}) and highly efficient blue-light TADF materials. When the thermally activated delayed fluorescent compounds are used as a light-emitting material for an organic light-emitting display device, and the organic light-emitting display device is improved to have high luminous efficiency.

### BACKGROUND OF INVENTION

### Field of Invention

The present invention relates to a field of electroluminescent materials, and particularly, to a thermally activated delayed fluorescence material, a method for manufacturing thereof, and an organic electroluminescent diode device.

### Description of Prior Art

It is known that organic light-emitting diodes (OLEDs) have active light emission without a backlight, and the OLEDs have advantages of high luminous efficiency, wide viewing angles, fast response times, a wide tolerance of temperature, relatively simple processing techniques, a low driving voltage, low energy consumption, being lighter and thinner, a flexible display, and good future application prospects, and thus many researchers focus on OLEDs.

The principle of OLED devices is that, holes and electrons are injected from an anode and a cathode, respectively, under an electric field, and the holes and electrons pass through a hole injection layer, a hole transport layer, an electron injection layer, and an electron transport layer. Excitons are formed in a light-emitting layer, and light will be emitted by radiation attenuation of the excitons.

Organic electroluminescent materials used as a core component of OLED devices have a great influence on the performance of devices. Moreover, dominant guest luminescent material is critical for the devices. Traditionally, the luminescent guest materials used in early OLEDs are fluorescence materials. Because the ratio of singlet and triplet excitons in the OLED is 1:3, the theoretical internal quantum efficiency (IQE) of fluorescent-based OLEDs is merely 25%. Therefore, application of fluorescent electroluminescent devices is greatly limited. The phosphorescent heavy-metal complexes can achieve 100% IQE by using singlet and triplet excitons simultaneously due to the spin-orbit coupling of heavy atoms. However, the used heavy-metals are precious metals, such as Ir and Pt, and the phosphorescent heavy-metal complexes served as blue light materials should be improved.

Pure organic thermally activated delayed fluorescence (TADF) materials have a lowest single-triplet level difference (ΔEST) which is relatively less than ever before through a suitable molecular design, so that triplet excitons can be transformed to a singlet state by reverse intersystem crossing (RISC) and then are illuminated when jumping to a ground state transition by radiation. Therefore, single and triplet excitons can be simultaneously used and also achieve 100% IQE.

As for TADF materials, a high reaction rate constant of reverse intersystem enthalpy constant (k_{RISC}) and a high photoluminescence quantum yield (PLQY) are necessary for fabricating highly efficient OLEDs. Currently, TADF materials with the features above are still relatively scarce as compared with heavy metal Ir complexes. As for two-color white light, efficient blue-light TADF materials are essential. Therefore, design and synthesis of blue-light TADF materials for applying to the field are significant.

### SUMMARY OF INVENTION

In one embodiment of the present invention, one object is to provide a thermally activated delayed fluorescence (TADF) material to include a high reaction rate constant of reverse intersystem enthalpy constant and a high photoluminescence quantum yield. It can be used as a light-emitting layer material of an organic electroluminescent diode device due to blue-light TADF material having remarkable characteristics.

In another embodiment of the present invention, one object is to provide a method for preparing a thermally activated delayed fluorescent compound, and the method is easy to operate and has a high yield of a target product.

In another embodiment of the present invention, one object is to provide an organic electroluminescent diode device using the above thermally activated delayed fluorescent compound as a light emitting layer material, thereby improving luminescence efficiency of the device.

In order to achieve the above object, a thermally activated delayed fluorescent compound, comprising a chemical structure of formula I:

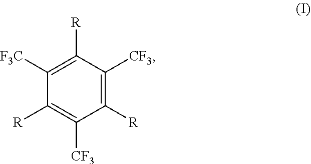

and R is an electron donating group.

The electron donating group R is selected any one of following groups:

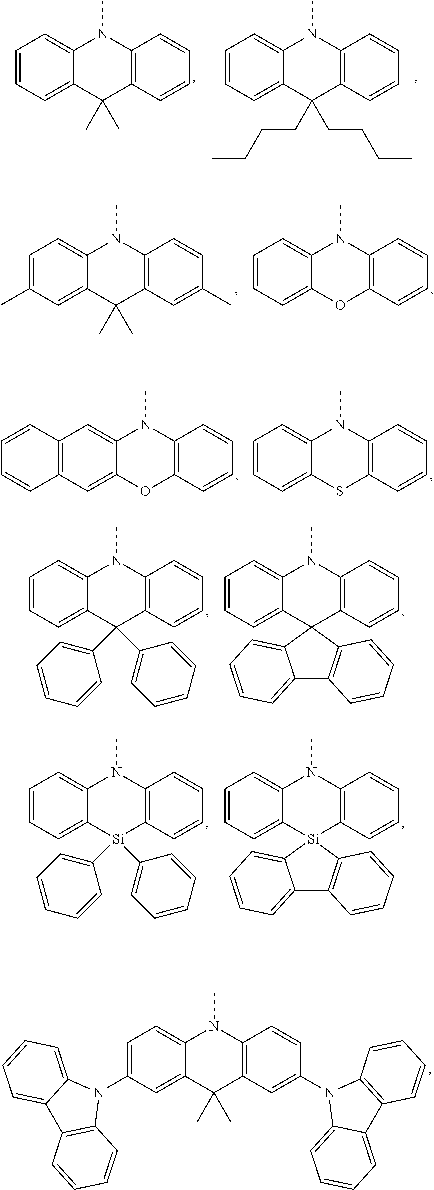

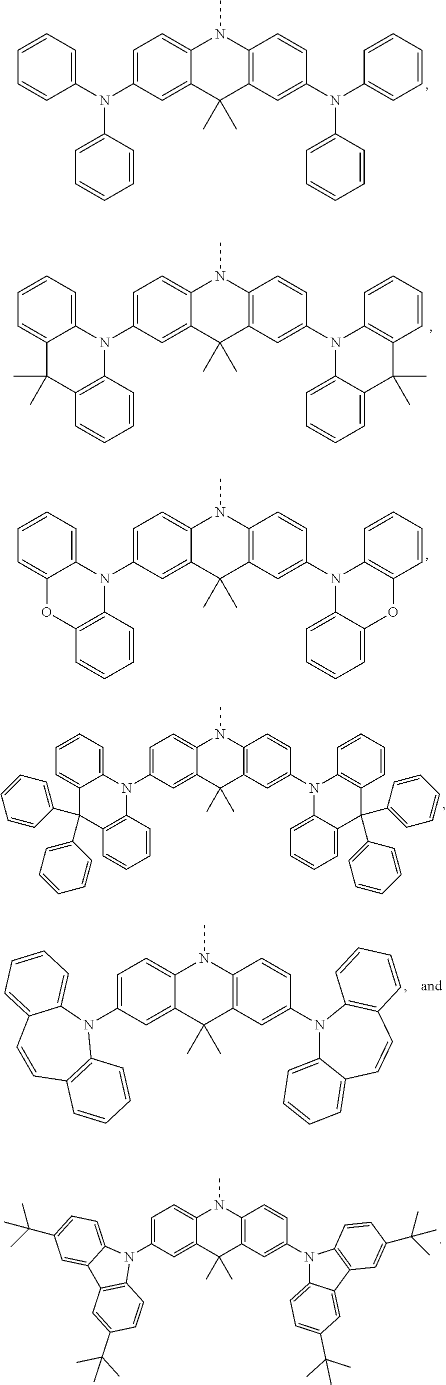

In one embodiment of the present invention, the thermally activated delayed fluorescent compound is compound 1, compound 2, or compound 3, and structure of formulas the compound 1, compound 2, and compound 3 are respectively represented as follows:

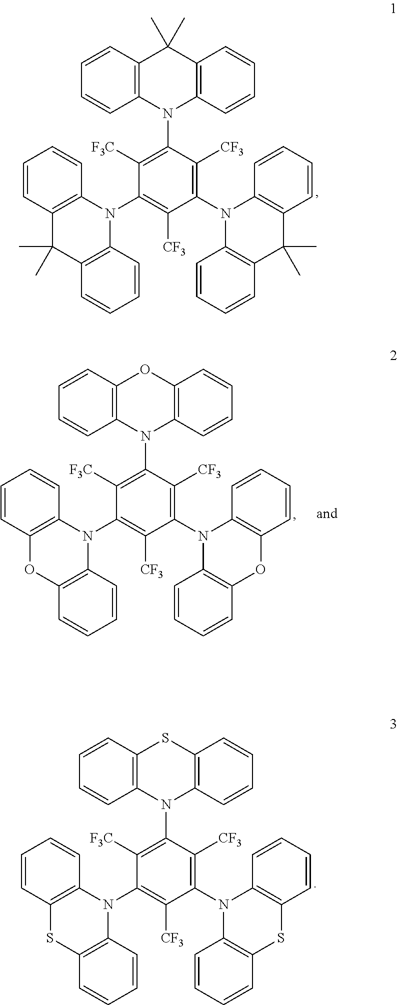

In another embodiment of the present invention, a method for preparing a thermally activated delayed fluorescent compound, a chemical reaction is presented as follows:

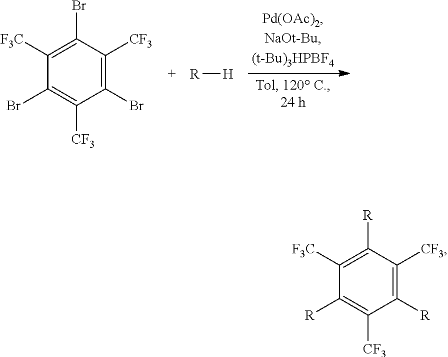

adding a raw material 1, an electron donor compound, palladium acetate and tri-tert-butylphosphine tetrafluoroborate to the reaction flask, and a molar ratio of the raw material, the electron donor compound, the palladium acetate, and the tri-tert-butylphosphine tetrafluoroborate is 1:3-4:0.1-0.2:0.3-0.4, and sodium tert-butoxide and raw material 1 are added to a glove box in a molar ratio of 3:4, and anhydrous, degassed toluene is added to the glove box under an argon atmosphere, and a reaction is performed at 120° C. for 24 hours, and reaction solution is cooled to room temperature and poured into ice water, and the reaction solution is extracted with dichloromethane for three times and combined with an organic phase, and the reaction solution is spun and dried, and the reaction solution is purified by column chromatography having a stationary phase of silica gel to obtain a product, and a yield is calculated.

A structural formula of the raw material 1 is

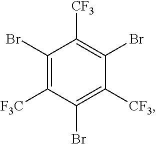 [SMILES: FC(F)(F)c1c(Br)c(C(F)(F)F)c(Br)c(C(F)(F)F)c1Br]

and a structural formula of an electron donating compound is represented as R—H, and R is represented as an electron donating group.

In one embodiment of the present invention, the electron donating group R is selected any one of following groups:

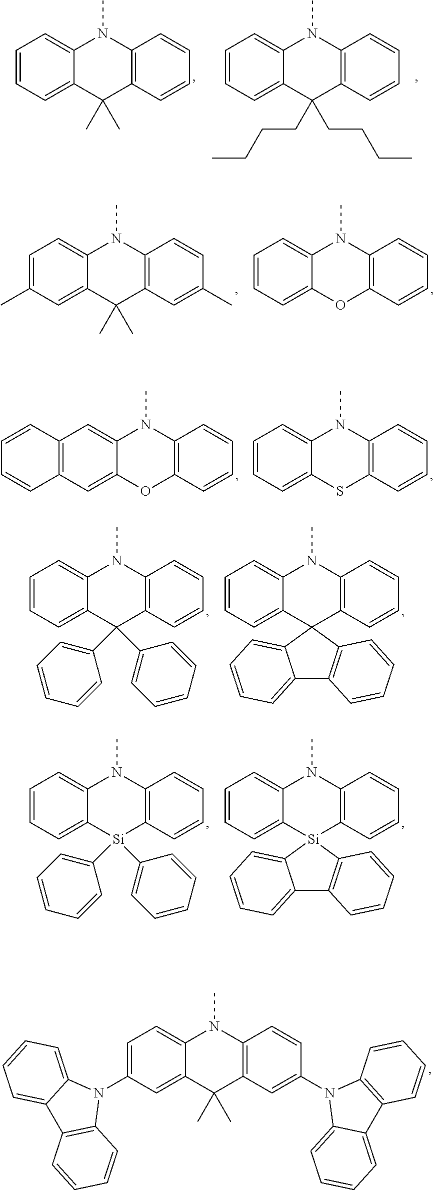

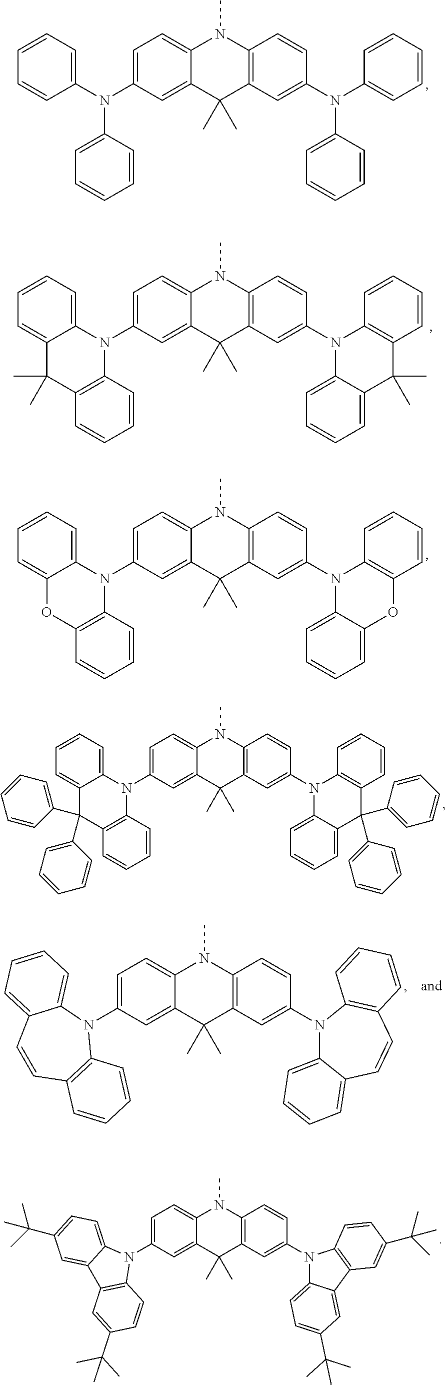

In one embodiment of the present invention, the electron donating compound is 9,10-dihydro-9,9-dimethyl acridine, phenoxazine or phenothiazine.

In another embodiment of the present invention, an organic electroluminescent diode device includes a substrate, a first electrode disposed on the substrate, an organic functional layer disposed on the first electrode, and a second electrode disposed on the organic functional layer of the first electrode. The organic functional layer includes an organic film or a multilayer organic film, and at least one of organic film is a luminescent layer. The luminescent layer includes above thermally activated delayed fluorescent compounds.

In one embodiment of the present invention, the luminescent layer is formed by vacuum evaporation or solution coating.

In one embodiment of the present invention, a material of the luminescent layer is a mixture of a host material and a guest material, and the guest material is selected from one or more of above thermally activated delayed fluorescent compounds.

In one embodiment of the present invention, the substrate is a glass substrate, a material of the first electrode is indium tin oxide, and the second electrode is a two-layer composite structure made of a lithium fluoride layer and an aluminum layer. The organic functional layer comprises a multilayer organic film, and the multilayer organic film comprises a hole injection layer, a hole transport layer, a luminescent layer, and an electron transport layer. A material of the hole injection layer is molybdenum trioxide. A material of the hole transport layer is Tris(4-carbazoyl-9-ylphenyl)amine (TCTA). A material of the electron transport layer is 1,3,5-Tris(3-pyridyl-3-phenyl)benzene (Tm3PyPB). A material of the luminescent layer is a mixture of a host material and a guest material, and the host material is Bis(2-(diphenylphosphino)phenyl)ether oxide (DPEPO). The guest material is selected from one or more of above thermally activated delayed fluorescent compounds.

Compared with existing materials and technologies, embodiments the present invention have advantages and benefits described as follows:

A trifluoromethyl group is used as a strong electron acceptor group, and an electron donor group is modified by combining different functional groups. An influence of the strength of the electron donor on the material properties is researched to design a blue-light thermal activation delayed fluorescent compounds with significant TADF properties.

The thermally activated delayed fluorescent compounds provided by the embodiment of the present invention have a high reaction rate constant of reverse intersystem enthalpy constant (k_{RISC}) and highly efficient blue-light TADF materials. When the thermally activated delayed fluorescent compounds are used as a light-emitting material for an organic light-emitting display device, a luminous efficiency of the organic light-emitting display device is improved. Therefore, an organic electroluminescent device using the thermally activated delayed fluorescent compounds achieves very high luminous efficiency.

### BRIEF DESCRIPTION OF DRAWINGS

The technical solutions and beneficial effects will be described in the following detailed description and drawings of embodiments.

FIG. 1 is a distribution diagram of an electron level of the lowest unoccupied molecular orbital (LUMO) and an electron level of the highest occupied molecular orbital (HOMO) of compound 1, compound 2, and compound 3.

FIG. 2 is a photoluminescence spectrum of compounds 1-3 in a toluene solution at room temperature according to embodiments 1-3 of the present invention.

FIG. 3 is a schematic view of an organic electroluminescent device according one embodiment of the present invention.

### DETAILED DESCRIPTION OF PREFERRED EMBODIMENTS

Some of the unspecified raw materials used in the present invention are commercially available products. The preparation of some compounds will be described in the examples. The present invention will be further described in detail below with reference to specific embodiments, but the embodiments of the present invention are not limited thereto.

### Example 1

A synthetic route of target compound 1 is described as follows:

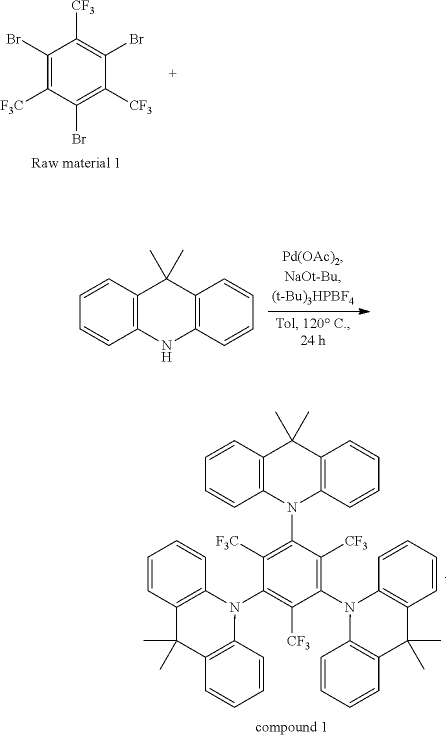

Raw material 1 (2.56 g, 5 mmol), 9,10-dihydro-9,9-dimethylacridine (3.76 g, 18 mmol), palladium acetate Pb(OAc) (135 mg, 0.6 mmol), and tri-tert-butylphosphine tetrafluoroborate (t-Bu)₃HPBF₄ (0.51 g, 1.8 mmol) are added to a 100 mL two-neck bottle, and then sodium tert-butoxide NaOt-Bu (1.74 g, 18 mmol) is added to a glove box. Anhydrous, degassed toluene (40 mL) is added to the glove box under an argon atmosphere. A reaction is performed at 120° C. for 24 hours. After cooling to room temperature, this reaction solution is poured into 200 mL of ice water. The reaction solution is extracted with dichloromethane for three times and combined with an organic phase. The reaction solution is spun and dried, and then the reaction solution is purified by column chromatography (Dichloromethane:Hexane, v:v, 2:1) having a stationary phase of silica gel to obtain a 3.0 g compound 1 which is a blue-white powder, and a yield is 66%. 1HNMR (300 MHz, CD2Cl2, δ): 7.19-7.14 (m, 18H), 6.95 (d, J=6.9 Hz, 6H), 1.69 (s, 18H)ₒ MS (EI) m/z: [M]+ calcd for C₅₄H₄₂F₉N₃, 903.32. found, 903.27.

### Example 2

A synthetic route of target compound 2 is described as follows:

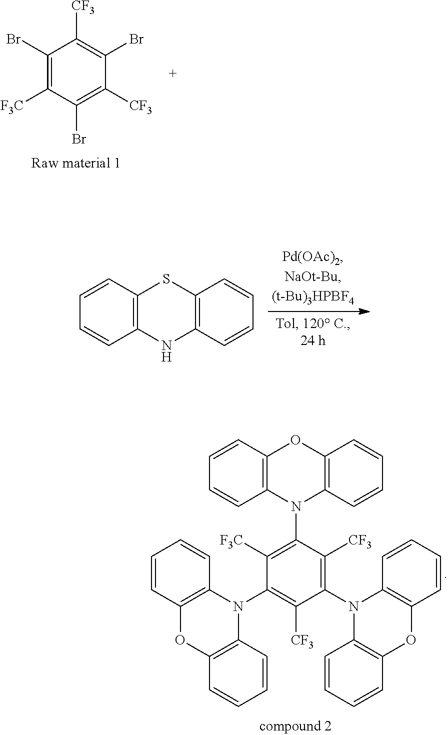 [SMILES: FC(F)(F)c1c(Br)c(C(F)(F)F)c(Br)c(C(F)(F)F)c1Br ; c1ccc2c(c1)Nc1ccccc1S2 ; FC(F)(F)c1c(N2c3ccccc3Oc3ccccc32)c(C(F)(F)F)c(N2c3ccccc3Oc3ccccc32)c(C(F)(F)F)c1N1c2ccccc2Oc2ccccc21]

Raw material 1 (2.56 g, 5 mmol), phenoxazine (3.30 g, 18 mmol), palladium acetate Pb(OAc) (135 mg, 0.6 mmol), and tri-tert-butylphosphine tetrafluoroborate (0.51 g, 1.8 mmol) are added to a 100 mL two-neck bottle, and then sodium tert-butoxide NaOt-Bu (1.74 g, 18 mmol) is added to a glove box. Anhydrous, degassed toluene (40 mL) is added to the glove box under an argon atmosphere. A reaction is performed at 120° C. for 24 hours. After cooling to room temperature, this reaction solution is poured into 200 mL of ice water. The reaction solution is extracted with dichloromethane for three times and combined with an organic phase. The reaction solution is spun and dried, and then the reaction solution is purified by column chromatography (Dichloromethane:Hexane, v:v, 2:1) having a stationary phase of silica gel to obtain a 2.7 g compound 2 which is a blue-white powder, and a yield is 65%. 1H NMR (300 MHz, CD2Cl2, δ): 7.14 (d, J=7.2 Hz, 6H), 7.01-6.96 (m, 18H). MS (EI) m/z: [M]+ calcd for C₄₅H₂₄F₉N₃O₃, 825.17. found, 825.13.

### Example 3

The synthetic route of target compound 3 is described as follows:

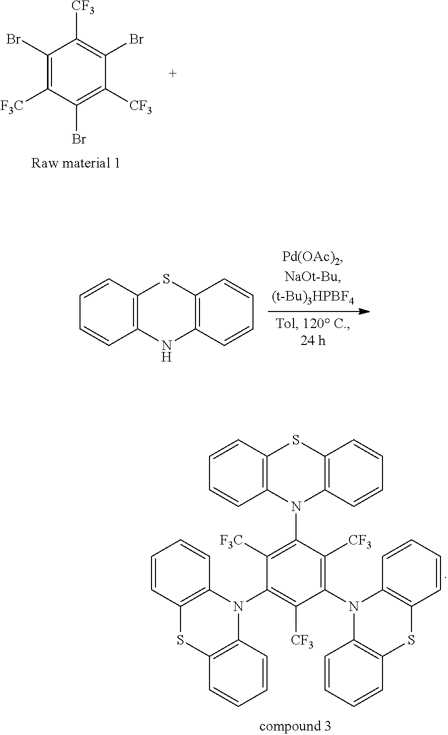 [SMILES: FC(F)(F)c1c(Br)c(C(F)(F)F)c(Br)c(C(F)(F)F)c1Br ; c1ccc2c(c1)Nc1ccccc1S2 ; FC(F)(F)c1c(N2c3ccccc3Sc3ccccc32)c(C(F)(F)F)c(N2c3ccccc3Sc3ccccc32)c(C(F)(F)F)c1N1c2ccccc2Sc2ccccc21]

Raw material 1 (2.56 g, 5 mmol), phenothiazine (3.30 g, 18 mmol), palladium acetate Pb(OAc) (135 mg, 0.6 mmol), and tri-tert-butylphosphine tetrafluoroborate (0.51 g, 1.8 mmol) are added to a 100 mL two-neck bottle, and then sodium tert-butoxide NaOt-Bu (1.74 g, 18 mmol) is added to a glove box. Anhydrous, degassed toluene (40 mL) is added to the glove box under an argon atmosphere. A reaction is performed at 120° C. for 24 hours. After cooling to room temperature, this reaction solution is poured into 200 mL of ice water. The reaction solution is extracted with dichloromethane for three times and combined with an organic phase. The reaction solution is spun and dried, and then the reaction solution is purified by column chromatography (Dichloromethane:Hexane, v:v, 2:1) having a stationary phase of silica gel to obtain a 2.8 g compound 3 which is a blue-white powder, and a yield is 64%. ¹H NMR (300 MHz, CD₂Cl₂, δ): 7.16-7.08 (m, 12H), 7.04-6.98 (m, 12H). MS (EI) m/z: [M]⁺ calcd for C₄₅H₂₄F₉N₃S₃, 873.10. found, 873.00.

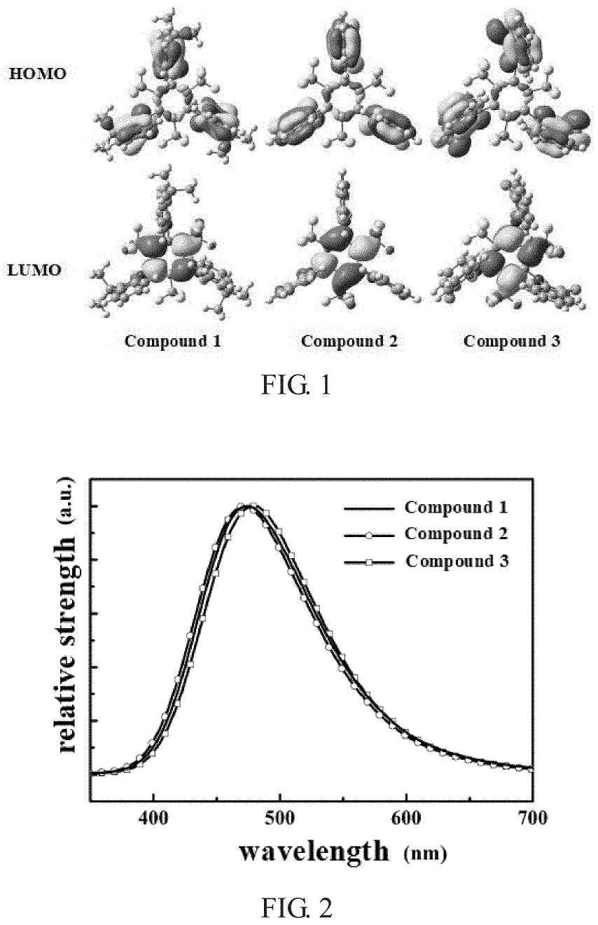

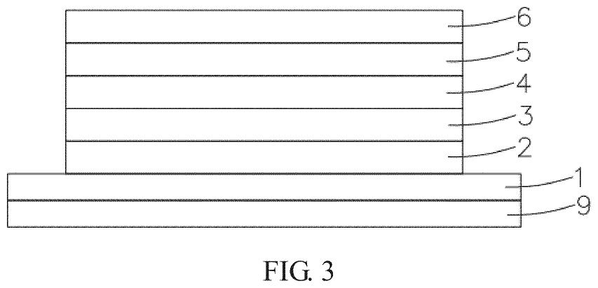

FIG. 1 is an orbital distribution diagram of compound 1, compound 2, and compound 3. As shown in FIG. 1, an electron level of the highest occupied molecular orbital (HOMO) and an electron level of the lowest unoccupied molecular orbital (LUMO) of compound 1, compound 2, and compound 3 are individually arranged on different units, and thus HOMO and LUMO of compound 1, compound 2, and compound 3 are completely separated. Therefore, the intersystem energy difference (ΔEST) is reduced, thereby improving a reverse intersystem crossing ability. FIG. 2 is a photoluminescence spectrum of compounds 1-3 in a toluene solution at room temperature. As for the compounds 1-3, the lowest singlet energy level S1 and the lowest triplet energy level T1 of the molecule are calculated by simulation.

The relevant data of Examples 1-3 are shown in Table 1. It can be seen from Table 1 that all compounds have a ΔEst of less than 0.3 ev, thereby achieving a less singlet energy level and triplet energy level difference with a significant delayed fluorescence effect.

TABLE 1
Photophysical properties of compounds 1-3

| | PL | | | | | |
|---|---|---|---|---|---|---|
| | Peak | S1 | T1 | ΔEST | HOMO | LUMO |
| | (nm) | (eV) | (eV) | (eV) | (eV) | (eV) |
| Compound 1 | 474 | 2.62 | 2.57 | 0.05 | −5.42 | −2.28 |
| Compound2 | 474 | 2.62 | 2.56 | 0.06 | −5.56 | −2.28 |
| Compound 3 | 477 | 2.60 | 2.54 | 0.06 | −5.58 | −2.28 |

In Table 1, PL Peak represents a photoluminescence peak. S1 represents a singlet energy level. T1 represents a triplet energy level. ΔEST represents a singlet and triplet energy level difference.

### Example 4

A method for fabricating an organic light-emitting diode (OLED) device is described as follows. Referring to FIG. 1, the thermally activated delayed fluorescent compounds according to embodiments of the present invention are used as a guest material of light-emitting layer in the organic light-emitting diode device. The organic light-emitting diode device includes a substrate 9, an anode layer 1, a hole injection layer 2, a hole transport layer 3, a light-emitting layer 4, an electron transport layer 5, and a cathode layer 6 that are disposed in order from bottom to top. The substrate 9 is a glass substrate, and the anode 1 is made of indium tin oxide (ITO). The substrate 9 and the anode 1 are configured to an ITO glass, and the ITO glass has a sheet resistance of is 10 Ω/cm². The hole injection layer 2 is made of molybdenum trioxide (MoO₃). The hole transport layer 3 is made of TCTA, and the light-emitting layer is made of a mixture of the activated delayed fluorescent compounds provided by the embodiments and DPEPO. The electron transport layer 5 is made of Tm3PyPB. The cathode is a two-layered structure composed of a lithium fluoride (LiF) layer and an aluminum (Al) layer. TCTA is 4,4′,4″-tris(carbazol-9-yl)triphenylamine, DPEPO is bis[2-((oxo)diphenylphosphino)phenyl]ether, and Tm3PyPB is 1,3, 5-tris(3-(3-pyridyl)phenyl)benzene.

Specifically, a method for fabricating the organic light-emitting diode device includes sequentially depositing a MoO₃ film (2 nm), a TCTA film (35 nm), a mixture of DPEPO and thermally activated delayed fluorescent compounds, a Tm3PyPB film (40 nm), a LiF film (1 nm), and an Al film (100 nm) on the clean ITO glass under high vacuum conditions. A device as shown in FIG. 1 is obtained by the method, and various specific device structures are described as follows:

Device 1: ITO/MoO₃ (2 nm)/TCTA (35 nm)/DPEPO: compound 1 (3% 40 nm)/TmPyPB (40 nm)/LiF (1 nm)/Al (100 nm)

Device 2: ITO/MoO₃ (2 nm)/TCTA (35 nm)/DPEPO: compound 2 (3% 40 nm)/TmPyPB (40 nm)/LiF (1 nm)/Al (100 nm)

Device 3: ITO/MoO₃ (2 nm)/TCTA (35 nm)/DPEPO: compound 3 (3% 40 nm)/TmPyPB (40 nm)/LiF (1 nm)/Al (100 nm)

Furthermore, each of the above devices 1-3 performances is measured, and current-brightness-voltage characteristics of the devices are achieved by a Keithley source measurement system (Keithley 2400 Sourcemeter, Keithley 2000 Currentmeter) with a silicon photodiode which is calibrated. An electroluminescence spectrum is measured by the French JY SPEX CCD3000 spectrometer, and all measurements are performed at room temperature in the atmosphere.

The data of each of the devices 1-3 are shown in Table 2:

TABLE 2

| | Maximum current | | Maximum external |
|---|---|---|---|
| | efficiency | | quantum efficiency |
| Device | (cd/A) | CIEy | (%) |
| Device 1 | 56.7 | 0.25 | 26.6 |
| Device 2 | 55.6 | 0.25 | 25.9 |
| Device 3 | 29.4 | 0.29 | 25.1 |

In Table 2, CIEy is the y-coordinate value in standard CIE color space.

In the above, the present application has been described in the above preferred embodiments, but the preferred embodiments are not intended to limit the scope of the invention, and a person skilled in the art may make various modifications without departing from the spirit and scope of the application. The scope of the present application is determined by claims.

What is claimed is:

1. A thermally activated delayed fluorescent compound, comprising a chemical structure of formula I: 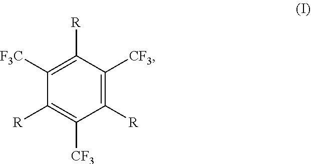
wherein R is an electron donating group.

2. The thermally activated delayed fluorescent compound according to claim 1, wherein the electron donating group R is selected any one of following groups:

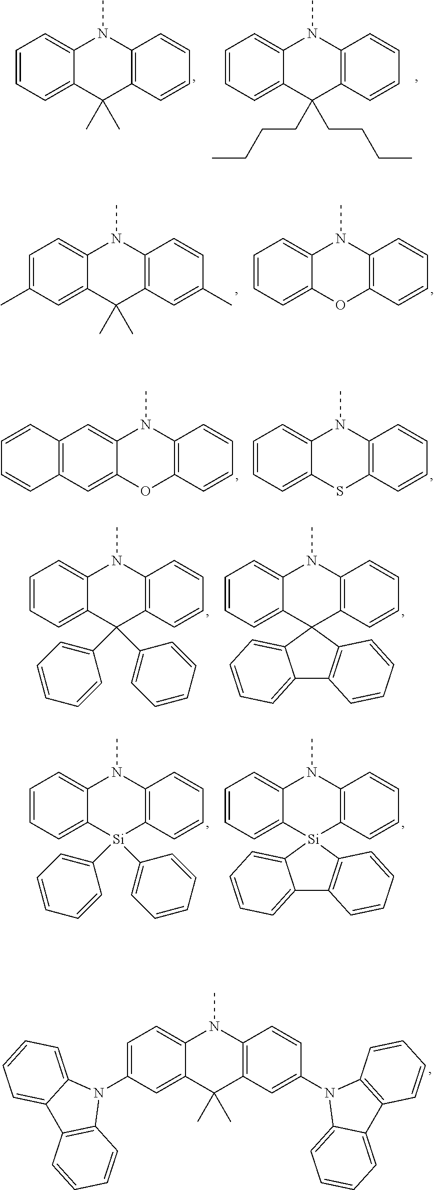

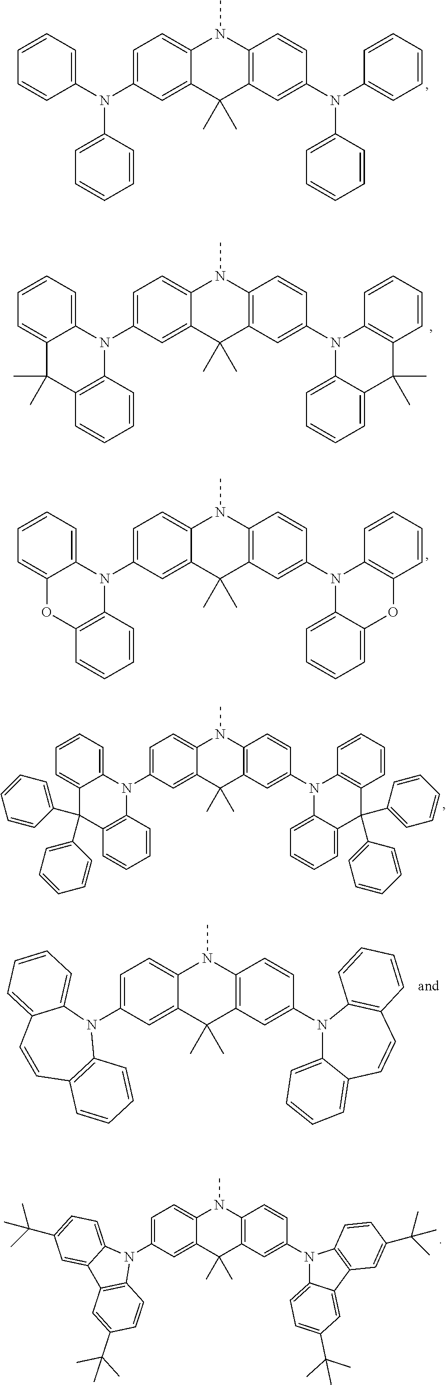

3. The thermally activated delayed fluorescent compound according to claim 2, wherein the thermally activated delayed fluorescent compound is compound 1, compound 2, or compound 3, and structure of formulas the compound 1, compound 2, and compound 3 are respectively represented as follows:

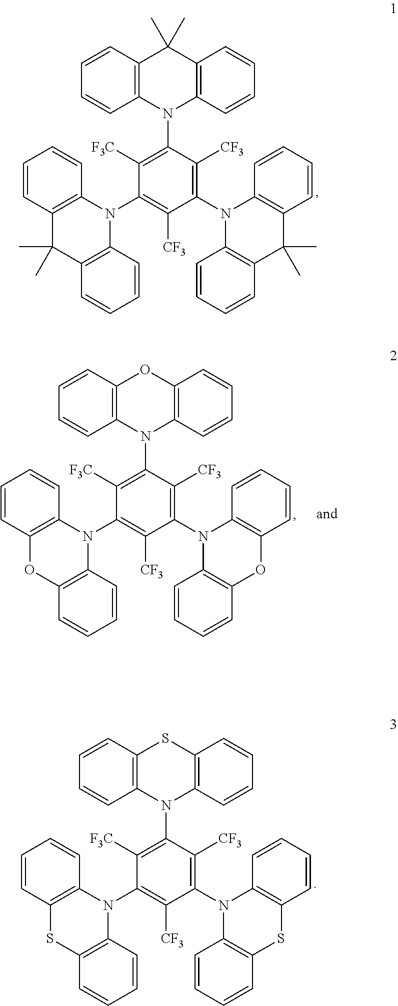

4. A method for preparing a thermally activated delayed fluorescent compound, a chemical reaction is presented as follows: 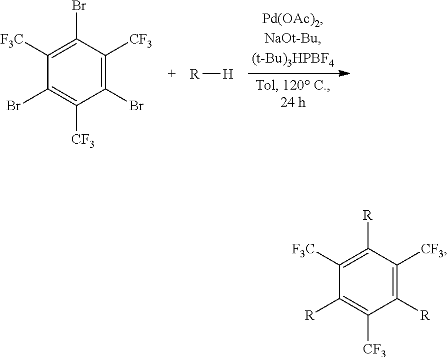
adding a raw material 1, an electron donor compound, palladium acetate, and tri-tert-butylphosphine tetrafluoroborate to a reaction flask, wherein a molar ratio of the raw material, the electron donor compound, the palladium acetate, and the tri-tert-butylphosphine tetrafluoroborate is 1:3-4:0.1-0.2:0.3-0.4, and sodium tert-butoxide and raw material 1 are added to a glove box in a molar ratio of 3:4, and anhydrous, degassed toluene is added to the glove box under an argon atmosphere, and a reaction is performed at 120° C. for 24 hours; wherein reaction solution is cooled to room temperature and poured into ice water, and the reaction solution is extracted with dichloromethane for three times and combined with an organic phase, and the reaction solution is spun and dried, and the reaction solution is purified by column chromatography having a stationary phase of silica gel to obtain a product, and a yield is calculated;
wherein a structural formula of the raw material 1 is 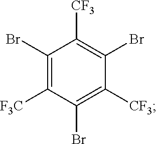 [SMILES: FC(F)(F)c1c(Br)c(C(F)(F)F)c(Br)c(C(F)(F)F)c1Br]
wherein a structural formula of an electron donating compound is represented as R—H,
and R is represented as an electron donating group.

5. The method for preparing the thermally activated delayed fluorescent compound according to claim 4, wherein the electron donating group R is selected any one of following groups:

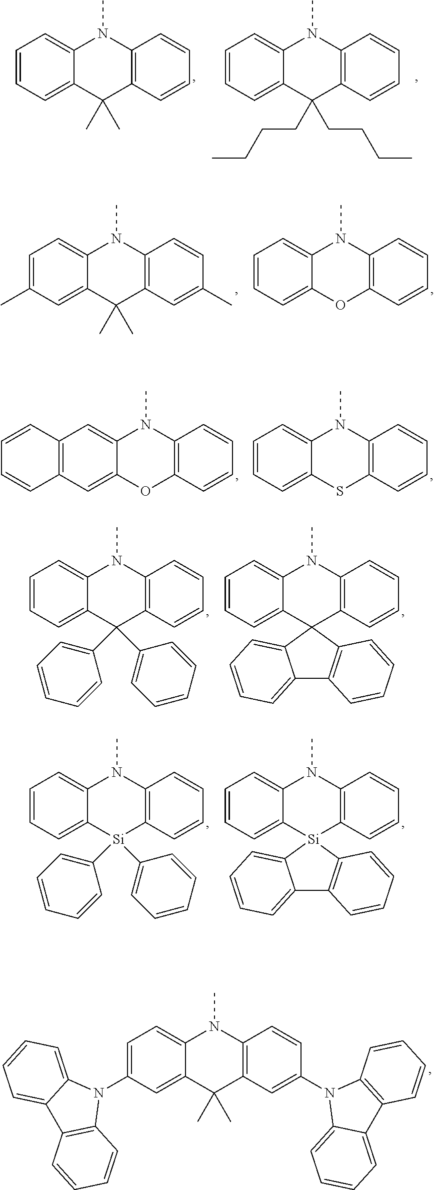

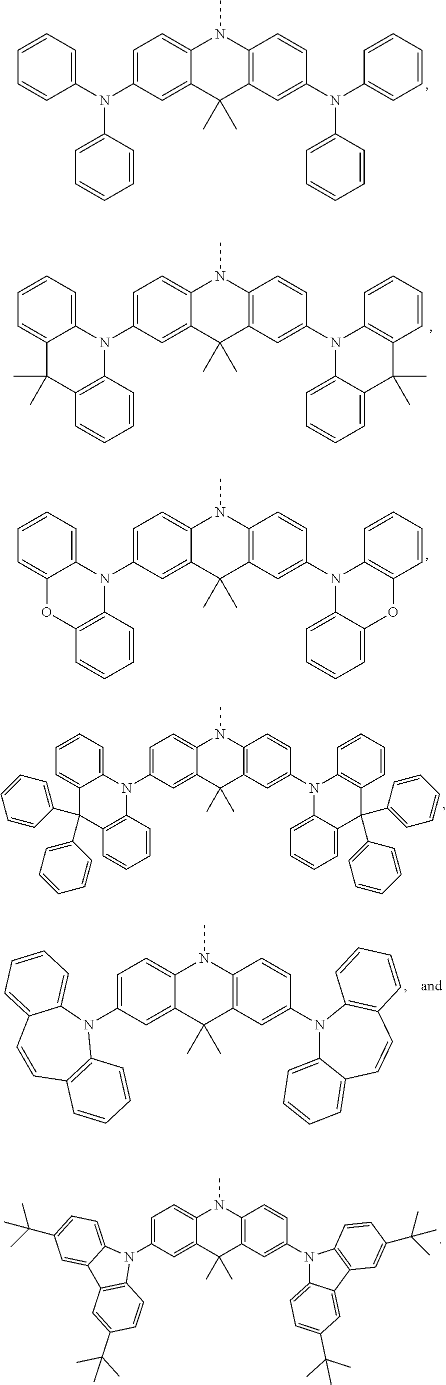

6. The method for preparing the thermally activated delayed fluorescent compound according to claim 5, wherein the electron donating compound is 9,10-dihydro-9,9-dimethyl acridine, phenoxazine or phenothiazine.

7. An organic electroluminescent diode device, comprising:
a substrate;
a first electrode disposed on the substrate;
an organic functional layer disposed on the first electrode;
and a second electrode disposed on the organic functional layer of the first electrode;
wherein the organic functional layer comprises an organic film or a multilayered organic film, and at least one of organic film is a luminescent layer; and
wherein luminescent layer comprises the thermally activated delayed fluorescent compound of claim 1.

8. The organic electroluminescent diode device according to claim 7, wherein the luminescent layer is formed by vacuum evaporation or solution coating.

9. The organic electroluminescent diode device according to claim 7, wherein a material of the luminescent layer is a mixture of a host material and a guest material, and the guest material is selected from one or more of the thermally activated delayed fluorescent compounds of claim 1.

10. The organic electroluminescent diode device according to claim 7, wherein the substrate is a glass substrate, a material of the first electrode is indium tin oxide, and the second electrode is a two-layered composite structure made of a lithium fluoride layer and an aluminum layer;
wherein the organic functional layer comprises a multilayered organic film, and the multilayered organic film comprises a hole injection layer, a hole transport layer, a luminescent layer, and an electron transport layer;
wherein a material of the hole injection layer is molybdenum trioxide;
wherein a material of the hole transport layer is Tris(4-carbazoyl-9-ylphenyl)amine (TCTA);
wherein a material of the electron transport layer is 1,3,5-Tris(3-pyridyl-3-phenyl)benzene (Tm3PyPB);
wherein a material of the luminescent layer is a mixture of a host material and a guest material, and the host material is Bis(2-(diphenylphosphino)phenyl)ether oxide (DPEPO); and
wherein the guest material is selected from one or more of the thermally activated delayed fluorescent compounds of claim 1.
